(19) United States
(12) Patent Application Publication
(10) Pub. No.: US 2026/0165571 A1
(43) Pub. Date: Jun. 18, 2026

(54) ENDOSCOPE REPROCESSOR, METHOD, AND CONTROL DEVICE FOR CONTROLLING FLUID PRESSURE IN ENDOSCOPE REPROCESSING

(71) Applicant: OLYMPUS MEDICAL SYSTEMS CORP., Tokyo (JP)

(72) Inventor: Kohei SEKINO, Tokyo (JP)

(73) Assignee: OLYMPUS MEDICAL SYSTEMS CORP., Tokyo (JP)

(21) Appl. No.: 19/533,570

(22) Filed: Feb. 9, 2026

Related U.S. Application Data
(63) Continuation of application No. PCT/JP2023/029254, filed on Aug. 10, 2023.

(51) Int. Cl. A61B 1/12; A61B 1/00

(52) U.S. Cl. CPC A61B 1/123; A61B 1/00128; A61B 1/125

(57) ABSTRACT

An endoscope reprocessor includes a first tube connectable to a first ferrule of an endoscope, a second tube connectable to a second ferrule of the endoscope, a fluid supply assembly, and a controller. The controller is configured to drive the fluid supply assembly to supply fluid to a conduit of the endoscope via the first and second tubes, and control a pressure of liquid supplied to a second conduit to be equal to or less than a pressure of liquid supplied to a first conduit. The conduit includes the first conduit in communication with the first ferrule, and the second conduit in communication with the second ferrule, the first and second conduits converging into a third conduit leading to an opening at a distal end of an insertion portion of the endoscope.

### CROSS REFERENCE TO RELATED APPLICATION

This application is a continuation of International Application No. PCT/JP2023/029254, filed on Aug. 10, 2023, which is incorporated herein by reference in its entirety.

### FIELD

The present disclosure relates to an endoscope reprocessor used for reprocessing process of endoscopes, a method of operating the endoscope reprocessor, and a control device.

### BACKGROUND

For endoscopes used in the medical field, reprocessing process, such as cleaning and disinfection, is essential for reuse after an insertion portion is inserted into a subject to perform intrasubject observation and treatment with a treatment instrument.

An endoscope reprocessor removes contaminants such as blood clots and mucus adhering to a conduit by connecting a connector of a tube to each of a suction ferrule and a forceps ferrule of an endoscope, supplying a fluid to each tube, and discharging the fluid from an opening at a distal end of an insertion portion.

International Publication No. WO2015-001843 discloses an endoscope cleaning apparatus that cleans a forceps ferrule using liquid that leaks out from a space between the forceps ferrule and a connector of a tube connected to the forceps ferrule.

International Publication No. WO2016-194456 discloses an endoscope reprocessor that performs flow control to adjust a flow rate of a first fluid supply unit that supplies liquid as a fluid and a flow rate of a second fluid supply unit that supplies gas as a fluid.

### SUMMARY

According to aspects of the present disclosure, an endoscope reprocessor is provided, which includes a first tube, a second tube, a fluid supply assembly, and a controller. The first tube is connectable to a first ferrule of an endoscope. The second tube is connectable to a second ferrule of the endoscope. The controller is configured to drive the fluid supply assembly to supply fluid to a conduit of the endoscope via the first and second tubes. The controller is further configured to control a pressure of liquid supplied to a second conduit to be equal to or less than a pressure of liquid supplied to a first conduit. The conduit includes the first conduit in communication with the first ferrule, and the second conduit in communication with the second ferrule, the first and second conduits converging into a third conduit leading to an opening at a distal end of an insertion portion of the endoscope.

According to aspects of the present disclosure, further provided is a method implementable on a controller of an endoscope reprocessor including a first tube, a second tube, and a fluid supply assembly. The method includes driving the fluid supply assembly to supply fluid to a conduit of an endoscope via the first and second tubes. The method further includes controlling a pressure of liquid supplied to a second conduit to be equal to or less than a pressure of liquid supplied to a first conduit. The endoscope includes an insertion portion, a first ferrule connectable to the first tube, a second ferrule connectable to the second tube, and the conduit including the first conduit in communication with the first ferrule, and the second conduit in communication with the second ferrule, the first and second conduits converging into a third conduit leading to an opening at a distal end of the insertion portion.

According to aspects of the present disclosure, further provided is a control device that includes a connection interface and a processor. The connection interface is connectable to a fluid supply assembly. The processor is configured to drive the fluid supply assembly to supply fluid to a conduit of an endoscope via a first tube and a second tube. The processor is further configured to control a pressure of liquid supplied to a second conduit to be equal to or less than a pressure of liquid supplied to a first conduit. The endoscope includes an insertion portion, a first ferrule, a second ferrule, and the conduit. The first ferrule is connectable to the first tube. The second ferrule is connectable to the second tube. The conduit includes the first conduit in communication with the first ferrule, and the second conduit in communication with the second ferrule, the first and second conduits converging into a third conduit leading to an opening at a distal end of the insertion portion.

### BRIEF DESCRIPTION OF DRAWINGS

FIG. 1 is a perspective view of an endoscope reprocessor according to a first embodiment.

FIG. 2 is a configuration diagram of a main part of the endoscope reprocessor according to the first embodiment.

FIG. 3 is a cross-sectional view of a joint section between a first connector and a forceps ferrule in the endoscope reprocessor according to the first embodiment.

FIG. 4 is a cross-sectional view of a convergence section of conduits in the endoscope reprocessor according to the first embodiment.

FIG. 5 is a flowchart for describing an example of a flow of cleaning processes for the endoscope reprocessor of the first embodiment.

FIG. 6 is a flowchart for describing an example of a flow of cleaning processes for the endoscope reprocessor of a second embodiment.

FIG. 7 is a flowchart for describing an example of a flow of cleaning processes for the endoscope reprocessor of a third embodiment.

FIG. 8 is a configuration diagram of a main part of an endoscope reprocessor according to a fourth embodiment.

FIG. 9 is a configuration diagram of a main part of an endoscope reprocessor according to a fifth embodiment.

FIG. 10 is a configuration diagram of a main part of an endoscope reprocessor according to a sixth embodiment.

### DESCRIPTION OF EMBODIMENTS

An endoscope reprocessor 1 of embodiments of the present disclosure will be described below using the drawings. Hereinafter, the endoscope reprocessor 1 will be referred to as a reprocessor 1.

It should be noted that the drawings according to the embodiment are schematic. The relationship between the thickness and width of each part and the ratio of the thickness of each part differ from the actual ones. The drawings also contain parts in which dimensional relationships and ratios are different from each other.

### First Embodiment

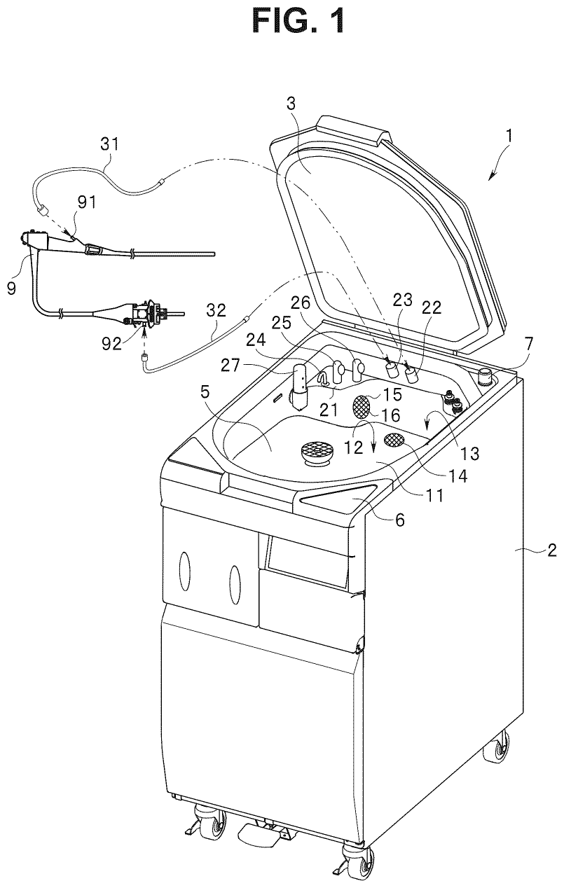

FIG. 1 is a perspective view of an endoscope reprocessor according to a first embodiment. As shown in FIG. 1, the reprocessor 1 includes a main body 2 and a top cover 3 which is openable and closable. FIG. 1 shows a state where the top cover 3 of the reprocessor 1 is open.

The reprocessor 1 is an apparatus for performing a restoration process on an endoscope 9 or an endoscope accessory. The restoration process may be rinsing with water, cleaning to eliminate contaminants such as organic matter, disinfection to inactivate predetermined microorganisms, sterilization to eliminate or kill all microorganisms, or any combination of these.

The main body 2 includes, at the top thereof, a processing tank 5 for performing a process such as cleaning and disinfection of the endoscope 9, an operation panel 6, and a water-supply-hose connection port 7.

The processing tank 5 stores a liquid such as a cleaning solution, water, an alcohol disinfectant solution, or a sterilization solution. The processing tank 5 includes an endoscope placement section 11 and a terrace 21.

The endoscope placement section 11 includes a bottom surface 12 and a side surface 13, is capable of placing the endoscope 9 thereon, and stores liquid. A discharge port 14 for discharging the stored liquid is provided in the bottom surface 12 of the endoscope placement section 11. A circulation port 16 having a mesh filter 15 is provided on the side surface 13 of the endoscope placement section 11. The circulation port 16 is in communication with a liquid pump 51 mentioned later. The circulation port 16 may be provided on the bottom surface 12.

The terrace 21 is provided at a position adjacent to the endoscope placement section 11 and higher than the endoscope placement section 11. The terrace 21 includes a forceps plug port 22 which is a first tube connection port, a suction ferrule port 23 which is a second tube connection port, a cleaning agent nozzle 24, a disinfectant solution nozzle 25, a water supply nozzle 26, and a water level sensor 27.

The forceps plug port 22 is a port to which a first tube 31 is to be connected. The suction ferrule port 23 is a port to which a second tube 32 is to be connected. The first tube 31 and the second tube 32 configure a cleaning tube. The number of ports that the reprocessor 1 has is not limited to 2.

The cleaning agent nozzle 24 supplies a cleaning solution into the processing tank 5. The disinfectant solution nozzle 25 supplies a disinfectant solution into the processing tank 5. The water supply nozzle 26 supplies water taken in from the water-supply-hose connection port 7 into the processing tank 5, and also supplies the liquid in the processing tank 5 taken in from the circulation port 16 having the mesh filter 15 back into the processing tank 5, thereby circulating the liquid. The mesh filter 15 filters contaminants P from the liquid. The water level sensor 27 detects a water level of the liquid stored in the processing tank 5.

The operation panel 6 is placed at a front section on top of the reprocessor 1. The operation panel 6 includes various operation buttons, not shown. A user gives various instructions to the reprocessor 1 via the operation panel 6.

The water-supply-hose connection port 7 is provided at a rear section on top of the reprocessor 1. A water supply hose connected to a water faucet, not shown, is connected to the water-supply-hose connection port 7, and supplies water into the reprocessor 1 via the water supply nozzle 26.

The top cover 3 is provided openably and closably on the top of the main body 2. In the reprocessor 1, by bringing the top cover 3 into an open state, the endoscope 9 can be placed on the endoscope placement section 11, and connection can be established between the endoscope 9 and the reprocessor 1 by means of the first tube 31 and the second tube 32. The reprocessor 1 is in a state where a process such as cleaning and disinfection is possible by setting the endoscope 9 and then bringing the top cover 3 into a closed state.

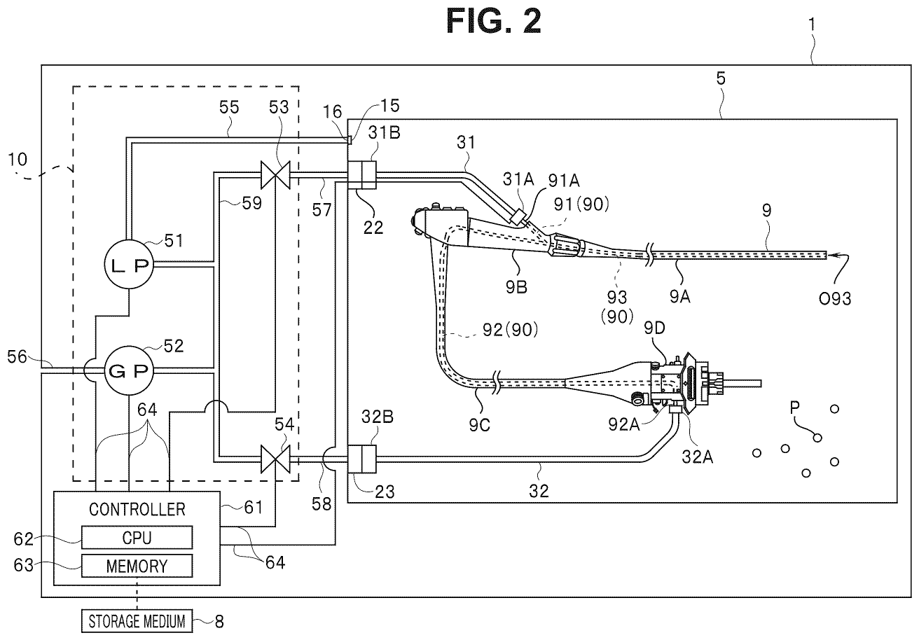

FIG. 2 is a configuration diagram of a main part of the endoscope reprocessor according to the first embodiment. FIG. 2 shows a state where the endoscope 9 to which the first tube 31 and the second tube 32 are connected is housed in the reprocessor 1. It should be noted that FIG. 2 shows only the main configuration of the present disclosure. The reprocessor 1 may have a configuration different from that shown in FIG. 2 as long as it has the same functions as the configuration shown in FIG. 2.

The endoscope 9 includes an insertion portion 9A to be inserted into a body, an operation portion 9B, a universal cord 9C, and an endoscope connector 9D. The endoscope 9 includes a conduit 90 inside thereof. The conduit 90 includes a first conduit 91, a second conduit 92, and a third conduit 93. One end of the first conduit 91 is provided with a forceps ferrule 91A, and the other end converges with the second conduit 92. One end of the second conduit 92 is provided with a suction ferrule 92A, and the other end converges with the first conduit 91.

The third conduit 93, where the first conduit 91 and the second conduit 92 converge, passes through the insertion portion 9A and has an opening O93 at a distal end. In other words, the conduit 90 includes the first conduit 91 and the second conduit 92 that converge into the third conduit 93 leading to the opening O93 at the distal end of the insertion portion 9A. A treatment instrument such as a forceps inserted from the forceps ferrule 91A passes through the first conduit 91 and the third conduit 93, and a distal end of the treatment instrument protrudes from the opening O93. A fluid fed from the suction ferrule 92A is emitted from the opening O93 via the second conduit 92 and the third conduit 93.

The first tube 31 includes a first connector 31A provided at one end thereof and connected to the forceps ferrule 91A which is a first ferrule of the endoscope 9, and a connector 31B provided at the other end thereof and connected to the forceps plug port 22 of the reprocessor 1. The forceps plug port 22 is in communication with the forceps ferrule 91A of the endoscope 9 via the first tube 31.

The second tube 32 includes a second connector 32A provided at one end thereof and connected to the suction ferrule 92A which is a second ferrule of the endoscope 9, and a connector 32B provided at the other end thereof and connected to the suction ferrule port 23 of the reprocessor 1. The suction ferrule port 23 is in communication with the suction ferrule 92A of the endoscope 9 via the second tube 32.

The reprocessor 1 includes the liquid pump 51, a gas pump 52, the processing tank 5, a first solenoid valve 53, a second solenoid valve 54, the forceps plug port 22, the suction ferrule port 23, and a controller 61.

The liquid pump 51 takes in a liquid, such as a cleaning solution, in the processing tank 5 from the circulation port 16 having the mesh filter 15 into a conduit 55, pressurizes the taken-in liquid at a predetermined pressure, and feeds the pressurized liquid to a branch conduit 59. Although not shown, a part of the liquid taken in from the circulation port 16 is fed to the processing tank 5 by another pump. The mesh filter 15 filters the contaminants P that flow down from the conduit 90 of the endoscope 9 and float in the liquid in the processing tank 5.

The liquid pump 51 is connected to the first solenoid valve 53 and the second solenoid valve 54 via the branch conduit 59. The first solenoid valve 53 is connected to the forceps plug port 22 via a conduit 57. The second solenoid valve 54 is connected to the suction ferrule port 23 via a conduit 58.

The gas pump 52 takes in gas via a conduit 56, pressurizes the taken-in gas at a predetermined pressure, and feeds the pressurized gas to the branch conduit 59. The gas is air, for example. The gas pump 52 is connected to the first solenoid valve 53 and the second solenoid valve 54 via the branch conduit 59.

The controller 61 includes a CPU 62, which is a central processing unit of a computer, and a memory 63 including a ROM, a RAM, or the like. Functions of the controller 61 are realized by the CPU 62 reading and executing a program from the memory 63. A program for causing the computer to execute a reprocessing process stored in the memory 63 is retained in a non-transitory computer-readable storage medium 8, and may be transferred to the memory 63.

The controller 61 further includes connection interfaces 64. The controller 61 is electrically connected to the liquid pump 51, the gas pump 52, the first solenoid valve 53, and the second solenoid valve 54 via respective connection interfaces 64. Each connection interface 64 may include an electrical line and a connector through which control signals are transmitted between the controller 61 and a respective one of the connected elements such as the liquid pump 51, the gas pump 52, the first solenoid valve 53, or the second solenoid valve 54.

When supplying liquid to the conduit 90, the controller 61 activates the liquid pump 51 and stops the gas pump 52. When supplying gas to the conduit 90, the controller 61 stops the liquid pump 51 and activates the gas pump 52.

When supplying a gas-liquid two-phase flow to the conduit 90, the controller 61 activates the liquid pump 51 and then activates the gas pump 52. A gas-liquid two-phase flow refers to a state in which liquid and gas are mixed in the conduit 90 of the endoscope 9 by feeding air from the gas pump 52 in a state where the conduit 90 is filled with liquid. More specifically, the gas-liquid two-phase flow includes any of a state in which gas bubbles are present in liquid, a state in which liquid droplets are present in the gas, and a state in which liquid slugs and gas pockets are present adjacent to one another.

The controller 61 controls an opening or closing state of the first solenoid valve 53 so that a fluid at a predetermined flow rate (predetermined pressure) is supplied to the first conduit 91 via the first tube 31. The controller 61 controls an opening or closing state of the second solenoid valve 54 so that a fluid at a predetermined flow rate is supplied to the second conduit 92 via the second tube 32.

The liquid pump 51, the gas pump 52, the first solenoid valve 53, and the second solenoid valve 54 configure a fluid supply unit 10. The controller 61 controls the timing at which the fluid supply unit 10 supplies fluid to the conduit 90. Hereinafter, the fluid supply unit 10 may be referred to as a “fluid supply assembly 10.”

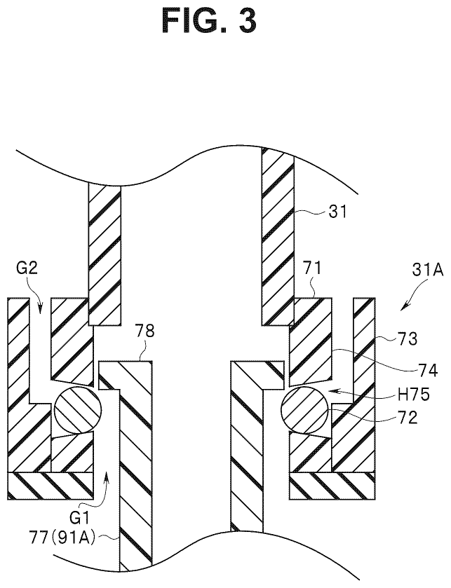

FIG. 3 is a cross-sectional view of a joint section between the first connector and the forceps ferrule in the endoscope reprocessor according to the first embodiment.

The first connector 31A includes a connector main body 71 provided at a distal end of the first tube 31, a plurality of spheres 72, and a connector cover 73 provided on an outer circumferential section of the connector main body 71.

The connector main body 71 is made of plastic or the like. The connector main body 71 is tubular, and includes a plurality of circular holes H75 in a circumferential side portion 74. For example, four holes H75 are provided in the circumferential side portion 74 of the connector main body 71 at equal intervals along a circumferential direction. Each hole H75 has a decreasing diameter from an outer surface toward an inner surface of the connector main body 71, and has a tapered cross section in a thickness direction of the circumferential side portion 74.

The plurality of spheres 72 is configured of metal or the like. The plurality of spheres 72 each has a diameter greater than that of the hole H75 in an inner circumferential surface of the circumferential side portion 74, and are placed with a part fitted in the holes H75, so as not to fall off from the inner circumferential surface of the circumferential side portion 74.

The connector cover 73 is configured of plastic or the like. The connector cover 73 is placed on an outside of the connector main body 71 where the plurality of spheres 72 is placed. A circumferential gap G2 is formed between the connector cover 73 and the connector main body 71.

The forceps ferrule 91A is made of metal, resin, or the like. The forceps ferrule 91A has a body section 77 formed in a cylindrical shape, and has an outwardly extending flange 78 at a distal end.

The first connector 31A of the first tube 31 is detachably attached to the forceps ferrule 91A. In a state where the first connector 31A is attached to the forceps ferrule 91A, the plurality (four, in this case) of spheres 72 of the first connector 31A engages with the outwardly extending flange 78 of the forceps ferrule 91A to inhibit detachment of the flange 78. A circumferential gap G1 is formed between the body section 77 of the forceps ferrule 91A and the connector main body 71.

The fluid fed from the first tube 31 is introduced into an inner side of the body section 77 of the forceps ferrule 91A, flows out from the gap G2 via a space between the connector main body 71 in which the hole H75 is formed and a sphere 72, and flows out from the gap G1 via a space between the connector main body 71 and the forceps ferrule 91A. The fluid flowing out from the gaps G1 and G2 in a connection area between the first connector 31A and the forceps ferrule 91A cleans the forceps ferrule 91A.

It should be noted that the shape of the first connector 31A of the first tube 31 is not limited to that mentioned above, and a connector disclosed in international publication WO2015-001843 already described can also be employed.

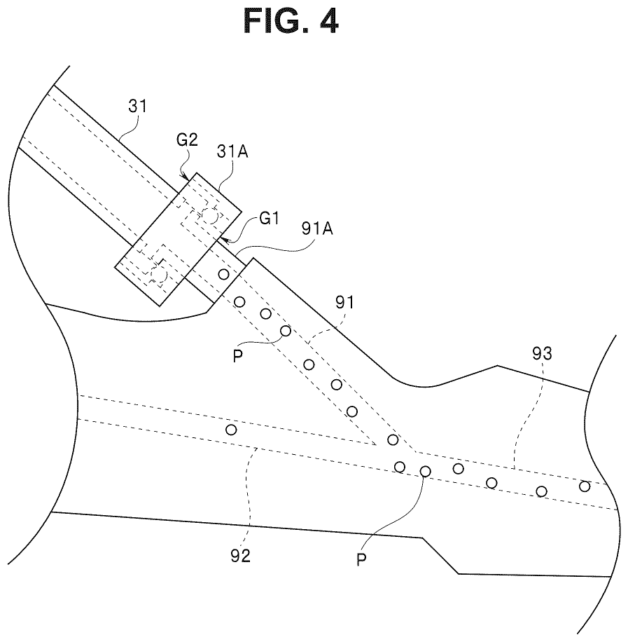

FIG. 4 is a cross-sectional view of a convergence section of the conduits in the endoscope reprocessor according to the first embodiment. As shown in FIG. 4, the contaminants P may adhere to the conduit 90 of the endoscope 9 after use, particularly to the first conduit 91 and the third conduit 93, which are withdrawal paths for the treatment instrument inserted into the body.

As already described, since the gaps G1 and G2 are present between the first connector 31A of the first tube 31 connected to the forceps ferrule 91A and the forceps ferrule 91A, if, in the reprocessing process, the fluid supplied to the second conduit 92 flows back through the first conduit 91 via the convergence section, the contaminants P flushed away by the fluid may be trapped in the gaps G1 and G2.

In the present embodiment, control is performed so that the pressure of liquid supplied to the second conduit 92 is equal to or less than the pressure of liquid supplied to the first conduit 91, thereby inhibiting backflow from the second conduit 92 to the first conduit 91. This inhibits the contaminants P from being trapped in the gaps G1 and G2.

Specifically, pressure loss of fluid is expressed by the following equation (1):

$$Δ P = 4 f \times {pu}^{2} / 2 \times L / d \qquad ( 1 )$$

where
ΔP is pressure loss [Pa],
f is friction coefficient,
p is fluid density [kg/m³],
u is average fluid velocity [m/s],
L is length of the conduit [m], and
d is diameter of the conduit [m].

From this equation (1), pressure loss is determined by the ratio of length to diameter of the conduit and fluid velocity. When liquid is fed by the fluid supply unit 10, which is a single driving source, fluid velocities in the first conduit 91 and the second conduit 92 are equivalent to each other, so the pressure loss is determined by the ratio of length to diameter of the conduit.

In the present embodiment, the first tube 31 and the second tube 32 are used that satisfy a relationship such that a ratio (L₁/d₁) of conduit length (L₁) to conduit diameter (d₁) of the first tube 31 and the first conduit 91 is equal to or less than a ratio (L₂/d₂) of conduit length (L₂) to conduit diameter (d₂) of the second tube 32 and the second conduit 92. In this way, control is performed so that the pressure of the liquid supplied to the second conduit 92 is equal to or less than the pressure of the liquid supplied to the first conduit 91.

Next, cleaning processes, which are operations of the endoscope reprocessor 1, will be described.

The user opens the top cover 3 of the endoscope reprocessor 1, and sets an endoscope 9 to be cleaned in the endoscope reprocessor 1. Specifically, the user connects the connector 31B of the first tube 31 to the forceps plug port 22, connects the first connector 31A to the forceps ferrule 91A of the endoscope 9, connects the connector 32B of the second tube 32 to the suction ferrule port 23, and connects the second connector 32A to the suction ferrule 92A of the endoscope 9. It should be noted that, although not shown, in addition to the connection by the first tube 31 and the second tube 32, a connection is established by tubes of the endoscope reprocessor 1 and the endoscope 9, as necessary.

After connecting the endoscope reprocessor 1 and the endoscope 9, the user places the endoscope 9 on the endoscope placement section 11 and brings the top cover 3 into the closed state.

When the user gives a start instruction for a process such as cleaning and disinfection via the operation panel 6, the CPU 62 reads a predetermined program from the memory 63 and starts the process of the program.

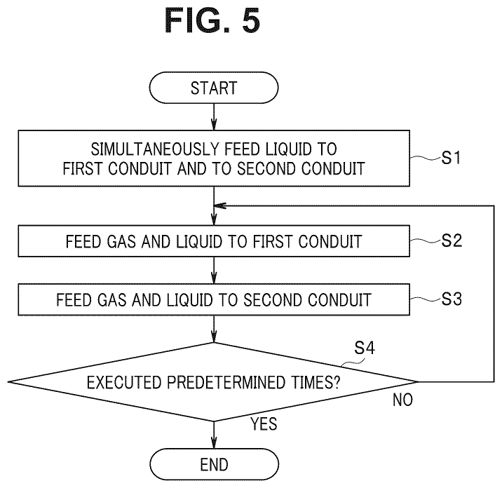

FIG. 5 is a flowchart for describing an example of a flow of the cleaning processes for the endoscope reprocessor of the first embodiment.

The controller 61 drives the fluid supply unit 10 to simultaneously feed liquid to the first conduit 91 and to the second conduit 92 (S1). By this process, the liquid fills the first conduit 91, the second conduit 92, and the third conduit 93 where the first conduit 91 and the second conduit 92 converge.

Subsequently, the controller 61 feeds gas and liquid to the first conduit 91 (S2). Next, the controller 61 feeds gas and liquid to the second conduit 92 (S3).

The controller 61 determines whether the cleaning processes in S2 and S3 have been executed a predetermined number of times (S4). If determining that the cleaning processes have not been executed the predetermined number of times (S4: NO), the controller 61 returns to the cleaning process in S2 and repeats the same processes. On the other hand, if determining that the cleaning processes have been executed the predetermined number of times (S4: YES), the controller 61 ends the cleaning processes.

As mentioned above, pressure loss of fluid is proportional to the conduit length and inversely proportional to the conduit diameter. For this reason, when the driving source for supplying fluid is the same (or has an equivalent output), the pressure loss is greater when the ratio of conduit length to conduit diameter is greater up to the convergence section where the first conduit 91 and the second conduit 92 converge.

The present embodiment has the relation that the ratio (L₁/d₁) of conduit length to conduit diameter of the first tube 31 and the first conduit 91 is equal to or less than the ratio (L₂/d₂) of conduit length to conduit diameter of the second tube 32 and the second conduit 92. For this reason, the fluid supplied to the second tube 32 and the second conduit 92 has a greater pressure loss.

For this reason, when liquid is fed by the fluid supply unit 10, which is a single driving source, a liquid feeding pressure to the first conduit 91 is higher than a liquid feeding pressure to the second conduit 92. As a result, the fluid supplied to the second conduit 92 does not flow back to the first conduit 91 via the convergence section, thus making it possible to inhibit the contaminants P flushed away by the fluid from being trapped in the gaps G1 and G2.

Since it is not necessary to perform control such as to stop feeding liquid to one of the first conduit 91 and second conduit 92 or to reduce the amount of feeding liquid to one of the conduits, it is possible to obtain a large total flow in a liquid feeding time that is equivalent to that in conventional cases.

Thus, according to the endoscope reprocessor 1 of the present embodiment, it is possible to suppress clogging of the space between the ferrule of the forceps port and the connector of the cleaning tube with contaminants, and to shorten the cleaning step time.

### Second Embodiment

Next, a second embodiment will be described.

It should be noted that the configuration of the reprocessor 1 is the same as that in the first embodiment, but the cleaning processes are different from those in the first embodiment.

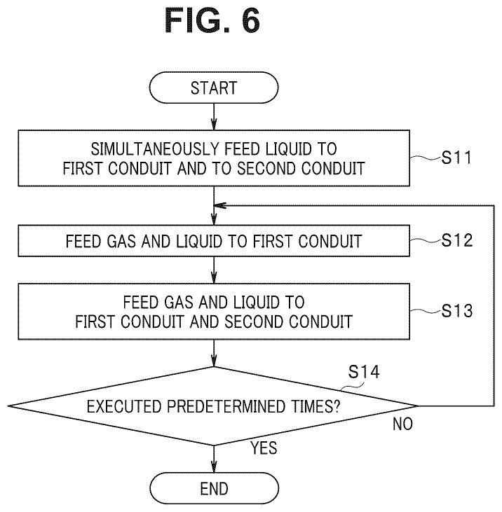

FIG. 6 is a flowchart for describing an example of a flow of cleaning processes for the endoscope reprocessor of the second embodiment.

The controller 61 drives the fluid supply unit 10 to simultaneously feed liquid to the first conduit 91 and to the second conduit 92 (S11). By this process, the liquid fills the first conduit 91, the second conduit 92, and the third conduit 93 where the first conduit 91 and the second conduit 92 converge.

Subsequently, the controller 61 feeds gas and liquid to the first conduit 91 (S12). Next, the controller 61 feeds gas and liquid to the first conduit 91 and the second conduit 92 (S13).

Then, the controller 61 determines whether the cleaning processes in S12 and S13 have been executed a predetermined number of times (S14). If determining that the cleaning processes have not been executed the predetermined number of times (S14: NO), the controller 61 returns to the cleaning process in S12 and repeats the same processes. On the other hand, if determining that the cleaning processes have been executed the predetermined number of times (S14: YES), the controller 61 ends the cleaning processes.

In the cleaning processes of the second embodiment, the total flow to the second conduit 92 is reduced compared to that in the cleaning processes of the first embodiment, but the reduction in total flow is not a problem in a configuration in which the second conduit 92 is easy to clean (e.g., including few complexly bent conduits and many straight conduits).

On the other hand, in the cleaning processes of the second embodiment, gas and liquid are always fed to the first conduit 91, which is a withdrawal path for the treatment instrument, thus making it possible to remove the contaminants P more effectively.

According to the endoscope reprocessor 1 of the present embodiment, as in the first embodiment, it is possible to suppress clogging of the space between the ferrule of the forceps port and the connector of the cleaning tube with contaminants, and to shorten the cleaning step time.

### Third Embodiment

Next, a third embodiment will be described.

It should be noted that the configuration of the reprocessor 1 is the same as that in the first embodiment, but the cleaning processes are different from those in the first embodiment.

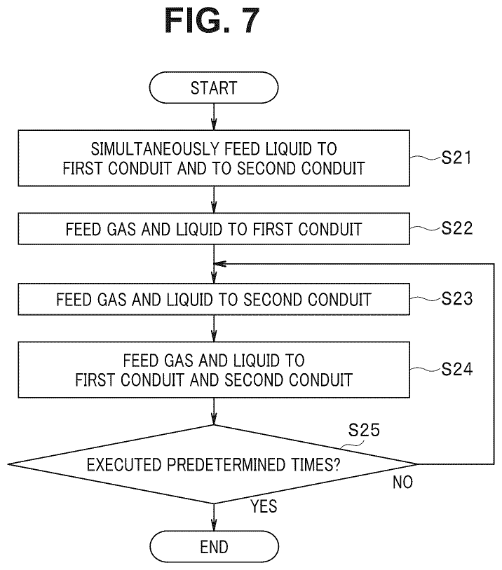

FIG. 7 is a flowchart for describing an example of a flow of cleaning processes for the endoscope reprocessor in the third embodiment.

The controller 61 drives the fluid supply unit 10 to simultaneously feed liquid to the first conduit 91 and to the second conduit 92 (S21). By this process, the liquid fills the first conduit 91, the second conduit 92, and the third conduit 93 where the first conduit 91 and the second conduit 92 converge.

Next, the controller 61 feeds gas and liquid to the first conduit 91 (S22). Subsequently, the controller 61 feeds gas and liquid to the second conduit 92 (S23). Next, the controller 61 feeds gas and liquid to the first conduit 91 and the second conduit 92 (S24).

Then, the controller 61 determines whether the cleaning processes in S23 and S24 have been executed a predetermined number of times (S25). If determining that the cleaning processes have not been executed the predetermined number of times (S25: NO), the controller 61 returns to the cleaning process in S23 and repeats the same processes. On the other hand, if determining that the cleaning processes have been executed the predetermined number of times (S25: YES), the controller 61 ends the cleaning processes.

In the cleaning processes of the third embodiment, it is configured such that, after feeding of gas and liquid to the first conduit 91 is carried out (S22), feeding of gas and liquid to the second conduit 92 is continued. With such cleaning processes, it is possible to efficiently perform cleaning processes on even the endoscope 9 in which the second conduit 92 has a configuration that is difficult to clean (e.g., including many complexly bent conduits and few straight conduits).

According to the endoscope reprocessor 1 of the present embodiment, as in the first embodiment, it is possible to suppress clogging of the space between the ferrule of the forceps port and the connector of the cleaning tube with contaminants, and to shorten the cleaning step time.

### Fourth Embodiment

Next, a fourth embodiment will be described.

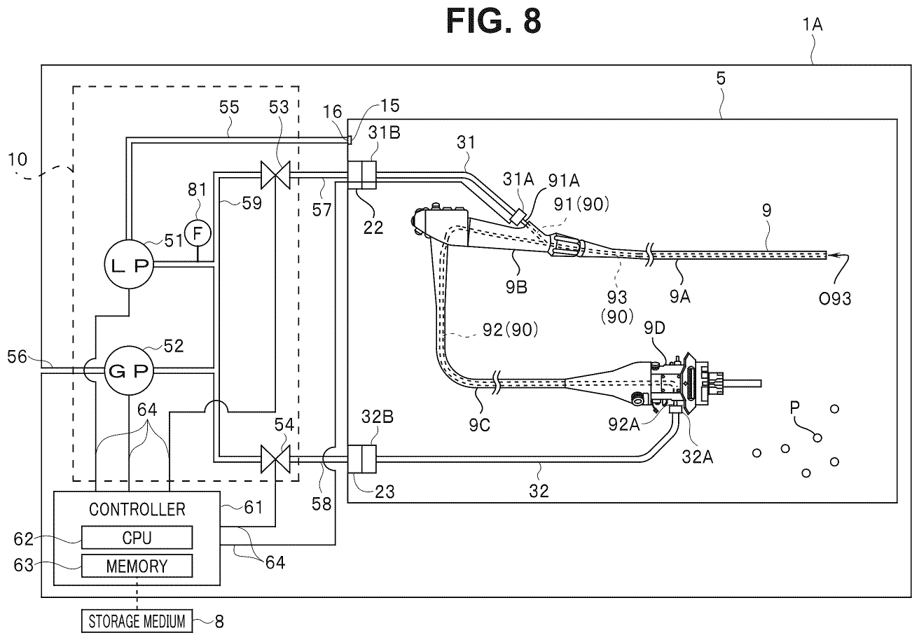

FIG. 8 is a configuration diagram of a main part of an endoscope reprocessor according to the fourth embodiment. It should be noted that, in FIG. 8, the same reference numerals are attached to the same configurations as in FIG. 2, and descriptions thereof will be omitted.

As shown in FIG. 8, an endoscope reprocessor 1A includes a flow sensor 81 disposed in a discharge conduit of the liquid pump 51, the flow sensor 81 detecting a total flow of liquid supplied to the first conduit 91 and/or the second conduit 92. A detection result of the flow sensor 81 configuring a flow detection section is outputted to the controller 61.

The controller 61 detects the total flow of liquid flowing to the conduit 90 based on the detection result of the flow sensor 81 to control, based on a volume from one ferrule to the other ferrule, the timing of switching the conduit for feeding gas and liquid, to thereby achieve more accurate inhibition of clogging.

Specifically, in the cleaning processes mentioned above, when switching from feeding gas and liquid to the second conduit 92 to feeding gas and liquid to the first conduit 91, the controller 61 detects the flow of gas and liquid fed to the second conduit 92 using the flow sensor 81, thereby determining a flow volume of gas and liquid fed to the second conduit 92.

The controller 61 then controls the fluid supply unit 10 to switch from feeding gas and liquid to the second conduit 92 to feeding gas and liquid to the first conduit 91 before the determined flow volume exceeds a capacity from the suction ferrule 92A to the forceps ferrule 91A.

As a result, the fluid supplied to the second conduit 92 does not flow back to the first conduit 91 via the convergence section, thus making it possible to inhibit the contaminants P flushed away by the fluid from being trapped in the gaps G1 and G2.

### Fifth Embodiment

Next, a fifth embodiment will be described.

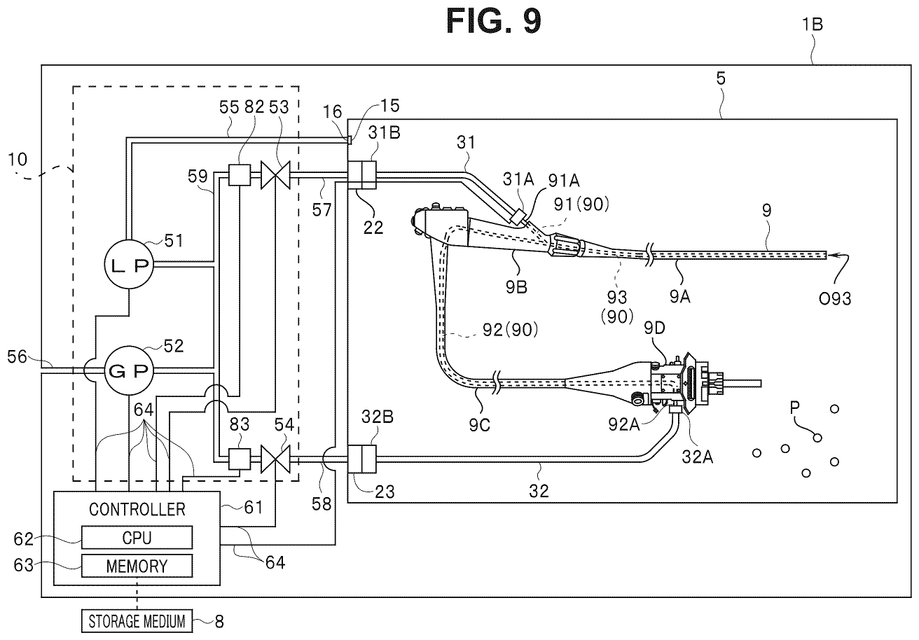

FIG. 9 is a configuration diagram of a main part of an endoscope reprocessor according to the fifth embodiment. It should be noted that, in FIG. 9, the same reference numerals are attached to the same configurations as in FIG. 2, and descriptions thereof will be omitted.

As shown in FIG. 9, an endoscope reprocessor 1B includes a pressure control section 82 disposed in the branch conduit 59 between the liquid pump 51 and the first solenoid valve 53. A pressure control section 83 is also disposed in the branch conduit 59 between the liquid pump 51 and the second solenoid valve 54. The pressure control sections 82 and 83 are electrically connected to the controller 61.

The pressure control sections 82 and 83 control the pressure of liquid supplied to the first conduit 91 and the pressure of liquid supplied to the second conduit 92 in accordance with a control signal from the controller 61.

It should be noted that the pressure control sections 82 and 83 may each be configured using a proportional valve or the like. When proportional valves are used as the pressure control sections 82 and 83, the first solenoid valve 53 and the second solenoid valve 54 can be omitted since these control sections can be opened and closed by the proportional valves. These proportional valves make the pressure loss in the second conduit 92 greater than the pressure loss in the first conduit 91, thereby making it possible to adjust the pressure difference between the first conduit 91 and the second conduit 92 at the convergence section.

For this reason, backflow into the first conduit 91 may be inhibited even in the endoscope 9 that is unable to obtain the relation that the ratio of conduit length to conduit diameter of the first tube 31 and the first conduit 91 is equal to or less than the ratio of conduit length to conduit diameter of the second tube 32 and the second conduit 92.

### Sixth Embodiment

Next, a sixth embodiment will be described.

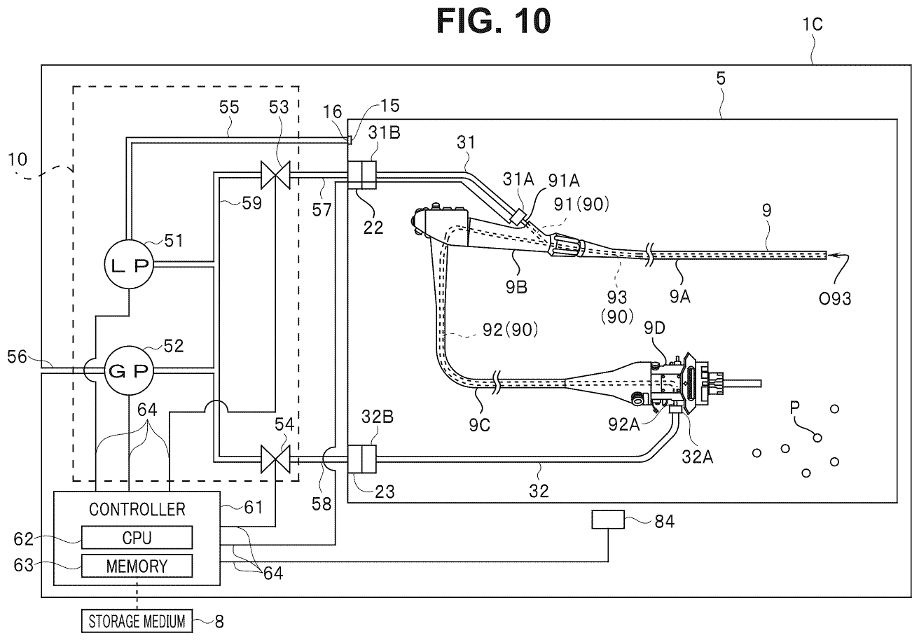

FIG. 10 is a configuration diagram of a main part of an endoscope reprocessor according to the sixth embodiment. It should be noted that, in FIG. 10, the same reference numerals are attached to the same configurations as in FIG. 2, and descriptions thereof will be omitted.

As shown in FIG. 10, an endoscope reprocessor 1C includes, adjacent to the processing tank 5, an acquisition section 84 that acquires endoscope information.

The acquisition section 84 includes an RF-ID reading section. RF-ID (Radio Frequency Identification) enables transmission and reception of information by wireless communication using electromagnetic fields or radio waves.

When an RF-ID, in which endoscope information related to the endoscope 9, such as the model name and serial number of the endoscope 9, is stored, is built into the endoscope 9, the acquisition section 84 acquires the endoscope information by the RF-ID reading section.

It should be noted that the acquisition section 84 may include, instead of or in addition to the RF-ID reading section, a camera that acquires an image of the endoscope 9 placed in the processing tank 5. The acquisition section 84 identifies the type of the endoscope 9 from the image of the endoscope 9 picked up by the camera, to thereby acquire the endoscope information.

The acquisition section 84 may include a user interface provided on the operation panel 6. The acquisition section 84 acquires the endoscope information from content inputted via this user interface. The user interface may be a physical operation button or an operation button displayed on a touch panel.

The conduit lengths and the conduit diameters of the first conduit 91 and the second conduit 92 of the endoscope 9 differ depending on the type of the endoscope 9. For this reason, by acquiring the endoscope information of the endoscope 9 by the acquisition section 84, it is possible to acquire information on the conduit lengths and the conduit diameters of the first conduit 91 and the second conduit 92 of the endoscope 9.

As a result, the user can select the first tube 31 and the second tube 32, in accordance with the type of the endoscope 9, so as to satisfy the relation that the ratio of conduit length to conduit diameter of the first tube 31 and the first conduit 91 is equal to or less than the ratio of conduit length to conduit diameter of the second tube 32 and the second conduit 92.

Furthermore, the controller 61 may switch the cleaning processes based on the endoscope information of the endoscope 9. For example, the controller 61 switches to the cleaning processes of the second embodiment when the first conduit 91 includes a configuration that is difficult to clean, and switches to the cleaning processes of the third embodiment when the second conduit 92 includes a configuration that is difficult to clean. In addition, when neither the first conduit 91 nor the second conduit 92 includes a configuration that is difficult to clean, the controller 61 switches to the cleaning processes of the first embodiment.

In this way, by switching the cleaning processes based on the endoscope information of the endoscope 9, it is possible to execute the optimal cleaning processes in accordance with the type of the endoscope 9 in which the first conduit 91 and the second conduit 92 have different shapes.

It should be noted that the steps in the flowchart in the present specification may, unless contrary to the nature thereof, be executed in a changed order, executed simultaneously, or executed in a different order for each execution.

The present disclosure is not limited to the above-mentioned embodiments, and it is needless to say that various modifications, combinations, and applications are possible without departing from the gist of the disclosure.

### General Interpretation Notes

The following applies throughout this specification and drawings.

It is noted that various connections are described between elements in the foregoing description. These connections, unless specified otherwise, may be either direct or indirect, and this specification is not intended to be limiting in that respect. Aspects of the present disclosure may be implemented using circuits (such as application-specific integrated circuits) or computer software stored on non-transitory computer-readable storage media, including but not limited to RAMs, ROMs, flash memories, EEPROMs, CD media, DVD media, temporary storage, hard disk drives, floppy drives, permanent storage, and the like.

As used herein, the term “processor” encompasses a single processor or a group of multiple processors, which may include a single-core processor, a multi-core processor, multiple processors within a single device, or multiple processors in wired or wireless communication with each other. Such processors may be locally or remotely distributed and may operate collaboratively or in a distributed fashion across a network of devices, the Internet, or the cloud to collectively perform the tasks attributed to the “processor” described herein. It should be understood that not all of the processors included in the system or device are necessarily involved in performing each operation attributed to the “processor.” Rather, only a subset of at least one processor may contribute to performing a particular operation. Furthermore, different subsets of at least one processor may contribute to performing different operations, and the composition of the subsets may vary from one operation to another. Similarly, the term “non-transitory computer-readable (storage) medium” encompasses a single storage medium or a group of multiple storage media, which may be locally or remotely distributed and may collectively store and provide access to instructions, data, or other information in a coordinated or distributed manner.

In the present disclosure, an inclusive OR—meaning that it includes either A, B, or both—may be expressed as “A and/or B,” “at least one of A or B,” or “at least one selected from the group consisting of A and B.” Additionally, the expressions “one of A or B” and “either A or B,” as used herein, refer to a case where A or B is selected exclusively, but not both. The same interpretation applies in cases where three or more selectable elements are considered.

Non-limiting examples according to aspects of the present disclosure will be described in the following clauses:

Clause 1: An endoscope reprocessor comprising:
a first tube including a first connector connected to a first ferrule of an endoscope;
a second tube including a second connector connected to a second ferrule of the endoscope;
a fluid supply unit that supplies fluid to a conduit of the endoscope via each of the first tube and the second tube; and
a controller that controls the fluid supply unit, wherein
the conduit includes a first conduit in communication with the first ferrule, a second conduit in communication with the second ferrule, and a third conduit where the first conduit and the second conduit converge, the third conduit including an opening at a distal end of an insertion portion of the endoscope, and
the controller controls the fluid supply unit so that a pressure of liquid supplied to the second conduit is equal to or less than a pressure of the liquid supplied to the first conduit.

Clause 2: The endoscope reprocessor according to clause 1, wherein the first tube and the first conduit have a relationship such that a ratio of conduit length to conduit diameter of the first tube and the first conduit is equal to or less than a ratio of conduit length to conduit diameter of the second tube and the second conduit.

Clause 3: The endoscope reprocessor according to clause 1, wherein the controller controls the fluid supply unit to feed the liquid to the first conduit and the second conduit, and then to feed the gas and the liquid, alternating between the first conduit and the second conduit.

Clause 4: The endoscope reprocessor according to clause 1, wherein the controller controls the fluid supply unit to feed the liquid to the first conduit and the second conduit, and then to feed the gas and the liquid, alternating between only the first conduit and both the first and second conduits.

Clause 5: The endoscope reprocessor according to clause 1, wherein the controller controls the fluid supply unit to feed the liquid to the first conduit and the second conduit, then to feed the gas and the liquid to the first conduit, and after the feeding of the gas and the liquid, to feed the gas and the liquid, alternating between only the second conduit and both the first and second conduits.

Clause 6: The endoscope reprocessor according to clause 3, further comprising:
a flow detection section that detects a total flow of the liquid, wherein
the controller controls the fluid supply unit to detect a total flow of the gas and the liquid to the first conduit based on a detection result of the flow detection section, and to switch from the feeding of the gas and the liquid to the first conduit to the feeding of the gas and the liquid to the second conduit before exceeding a capacity from the first ferrule to the second ferrule.

Clause 7: The endoscope reprocessor according to clause 6, wherein
the controller controls the fluid supply unit to detect a total flow of the gas and the liquid to the second conduit based on the detection result of the flow detection section, and to switch from the feeding of the gas and the liquid to the second conduit to the feeding of the gas and the liquid to the first conduit before exceeding a capacity from the second ferrule to the first ferrule.

Clause 8: The endoscope reprocessor according to clause 1, further comprising:
an acquisition section that acquires endoscope information of the endoscope.

Clause 9: The endoscope reprocessor according to clause 8, wherein the acquisition section includes an RF-ID reading section that reads an RF-ID provided on the endoscope, and acquires the endoscope information from the RF-ID.

Clause 10: The endoscope reprocessor according to clause 8, wherein the acquisition section includes a camera that acquires an image of the endoscope, and acquires the endoscope information from the image of the endoscope.

Clause 11: The endoscope reprocessor according to clause 8, wherein the acquisition section is a user interface provided on an operation panel, and acquires the endoscope information from content inputted via the user interface.

Clause 12: The endoscope reprocessor according to clause 8, wherein the controller switches a cleaning process based on the endoscope information acquired by the acquisition section,

Clause 13: The endoscope reprocessor according to clause 12, wherein
the controller, upon determining based on the endoscope information that neither the first conduit nor the second conduit includes a configuration that is difficult to clean, controls the fluid supply unit to feed the liquid to the first conduit and the second conduit, and then to feed the gas and the liquid, alternating between the first conduit and the second conduit.

Clause 14: The endoscope reprocessor according to clause 12, wherein
the controller, upon determining based on the endoscope information that the first conduit includes a configuration that is difficult to clean, controls the fluid supply unit to feed the liquid to the first conduit and the second conduit, and then to feed the gas and the liquid, alternating between only the first conduit and both the first and second conduits.

Clause 15: The endoscope reprocessor according to clause 12, wherein
the controller, upon determining based on the endoscope information that the second conduit includes a configuration that is difficult to clean, controls the fluid supply unit to feed the liquid to the first conduit and the second conduit, then to feed the gas and the liquid to the first conduit, and after the feeding of the gas and the liquid, to feed the gas and the liquid, alternating between only the second conduit and both the first and second conduits.

Clause 16: A method of operating an endoscope reprocessor, the method comprising performing a control with respect to an endoscope, wherein
the endoscope comprises:
a first conduit in communication with a first ferrule;
a second conduit in communication with a second ferrule; and
a third conduit where the first conduit and the second conduit converge, wherein the third conduit includes an opening at a distal end of an insertion portion of the endoscope, and
the control is such that a pressure of liquid supplied to the second conduit is equal to or less than a pressure of liquid supplied to the first conduit.

Clause 17: The method of operating an endoscope reprocessor according to clause 16, wherein the first tube and the first conduit have a relationship such that a ratio of conduit length to conduit diameter of the first tube and the first conduit is equal to or less than a ratio of conduit length to conduit diameter of the second tube and the second conduit.

Clause 18: The method of operating an endoscope reprocessor according to clause 16, wherein the control is such that the liquid is fed to the first conduit and the second conduit, and then the gas and the liquid are fed, alternating between the first conduit and the second conduit.

Clause 19: The method of operating an endoscope reprocessor according to clause 16, wherein the control is such that the liquid is fed to the first conduit and the second conduit, and then the gas and the liquid are fed, alternately between only the first conduit and both the first and second conduits.

Clause 20: A control device comprising a processor controlling with respect to an endoscope, wherein
the endoscope comprises:
a first conduit in communication with a first ferrule;
a second conduit in communication with a second ferrule; and
a third conduit where the first conduit and the second conduit converge, wherein the third conduit includes an opening at a distal end of an insertion portion of the endoscope, and
the control is such that a pressure of liquid supplied to the second conduit is equal to or less than a pressure of liquid supplied to the first conduit.

What is claimed is:

1. An endoscope reprocessor comprising:
a first tube connectable to a first ferrule of an endoscope;
a second tube connectable to a second ferrule of the endoscope;
a fluid supply assembly; and
a controller configured to:
drive the fluid supply assembly to supply fluid to a conduit of the endoscope via the first and second tubes; and
control a pressure of liquid supplied to a second conduit to be equal to or less than a pressure of liquid supplied to a first conduit,
wherein the conduit includes the first conduit in communication with the first ferrule, and the second conduit in communication with the second ferrule, the first and second conduits converging into a third conduit leading to an opening at a distal end of an insertion portion of the endoscope.

2. The endoscope reprocessor according to claim 1,
wherein the first tube and the first conduit have a relationship where a first ratio of a first conduit length to a first conduit diameter of a first path including the first tube and the first conduit is equal to or less than a second ratio of a second conduit length to a second conduit diameter of a second path including the second tube and the second conduit.

3. The endoscope reprocessor according to claim 1,
wherein the controller is further configured to control the fluid supply assembly to:
feed liquid to the first conduit and the second conduit; and
then alternate feeding gas and liquid to the first conduit and feeding gas and liquid to the second conduit.

4. The endoscope reprocessor according to claim 1,
wherein the controller is further configured to control the fluid supply assembly to:
feed liquid to the first conduit and the second conduit; and
then alternate feeding gas and liquid to the first conduit and feeding gas and liquid to the first conduit and the second conduit.

5. The endoscope reprocessor according to claim 1,
wherein the controller is further configured to control the fluid supply assembly to:
feed liquid to the first conduit and the second conduit;
then feed gas and liquid to the first conduit; and
thereafter alternate feeding gas and liquid to the second conduit and feeding gas and liquid to the first conduit and the second conduit.

6. The endoscope reprocessor according to claim 3, further comprising a flow detector configured to detect a flow of liquid,
wherein the controller is further configured to:
detect, based on a detection result of the flow detector, a flow of gas and liquid fed to the first conduit, thereby determining a flow volume of gas and liquid fed to the first conduit; and
control the fluid supply assembly to switch from feeding gas and liquid to the first conduit to feeding gas and liquid to the second conduit before the flow volume of gas and liquid fed to the first conduit exceeds a capacity between the first ferrule and the second ferrule.

7. The endoscope reprocessor according to claim 6,
wherein the controller is further configured to:
detect, based on the detection result of the flow detector, a flow of gas and liquid fed to the second conduit, thereby determining a flow volume of gas and liquid fed to the second conduit; and
control the fluid supply assembly to switch from feeding gas and liquid to the second conduit to feeding gas and liquid to the first conduit before the flow volume of gas and liquid fed to the second conduit exceeds the capacity between the second ferrule and the first ferrule.

8. The endoscope reprocessor according to claim 1, further comprising an acquisition section configured to acquire endoscope information of the endoscope.

9. The endoscope reprocessor according to claim 8,
wherein the acquisition section includes an RF-ID reader configured to read an RF-ID provided on the endoscope thereby acquiring the endoscope information from the RF-ID.

10. The endoscope reprocessor according to claim 8,
wherein the acquisition section includes a camera configured to capture an image of the endoscope, thereby acquiring the endoscope information from the image of the endoscope.

11. The endoscope reprocessor according to claim 8,
wherein the acquisition section includes a user interface provided on an operation panel, thereby acquiring the endoscope information from content entered via the user interface.

12. The endoscope reprocessor according to claim 8,
wherein the controller is further configured to switch between different types of cleaning processes based on the endoscope information acquired by the acquisition section.

13. The endoscope reprocessor according to claim 12,
wherein the controller is further configured to, in response to determining based on the endoscope information that neither the first conduit nor the second conduit includes a configuration that is difficult to clean, control the fluid supply assembly to:
feed liquid to the first conduit and the second conduit; and
then alternate feeding gas and liquid to the first conduit and feeding gas and liquid to the second conduit.

14. The endoscope reprocessor according to claim 12,
wherein the controller is further configured to, in response to determining based on the endoscope information that the first conduit includes a configuration that is difficult to clean, control the fluid supply assembly to:
feed liquid to the first conduit and the second conduit; and
then alternate feeding gas and liquid to the first conduit and feeding gas and liquid to the first conduit and the second conduit.

15. The endoscope reprocessor according to claim 12,
wherein the controller is configured to, in response to determining based on the endoscope information that the second conduit includes a configuration that is difficult to clean, control the fluid supply unit to:
feed liquid to the first conduit and the second conduit;
then feed gas and liquid to the first conduit; and
thereafter alternate feeding gas and liquid to the second conduit and feeding gas and liquid to the first conduit and the second conduit.

16. A method implementable on a controller of an endoscope reprocessor comprising a first tube, a second tube, and a fluid supply assembly, the method comprising:
driving the fluid supply assembly to supply fluid to a conduit of an endoscope via the first and second tubes; and
controlling a pressure of liquid supplied to a second conduit to be equal to or less than a pressure of liquid supplied to a first conduit,
wherein the endoscope comprises:
an insertion portion;
a first ferrule connectable to the first tube;
a second ferrule connectable to the second tube; and
the conduit including the first conduit in communication with the first ferrule, and the second conduit in communication with the second ferrule, the first and second conduits converging into a third conduit leading to an opening at a distal end of the insertion portion.

17. The method according to claim 16,
wherein the first tube and the first conduit have a relationship where a first ratio of a first conduit length to a first conduit diameter of a first path including the first tube and the first conduit is equal to or less than a second ratio of a second conduit length to a second conduit diameter of a second path including the second tube and the second conduit.

18. The method according to claim 16, further comprising controlling the fluid supply assembly to:
feed liquid to the first conduit and the second conduit; and
then alternate feeding gas and liquid to the first conduit and feeding gas and liquid to the second conduit.

19. The method according to claim 16, further comprising controlling the fluid supply assembly to:
feed liquid to the first conduit and the second conduit; and
then alternate feeding gas and liquid to the first conduit and feeding gas and liquid to the first conduit and the second conduit.

20. A control device comprising:
a connection interface connectable to a fluid supply assembly; and
a processor configured to:
drive the fluid supply assembly to supply fluid to a conduit of an endoscope via a first tube and a second tube; and
control a pressure of liquid supplied to a second conduit to be equal to or less than a pressure of liquid supplied to a first conduit,
wherein the endoscope comprises:
an insertion portion;
a first ferrule connectable to the first tube;
a second ferrule connectable to the second tube; and
the conduit including the first conduit in communication with the first ferrule, and the second conduit in communication with the second ferrule, the first and second conduits converging into a third conduit leading to an opening at a distal end of the insertion portion.
